(19) United States
(12) Patent Application Publication
(10) Pub. No.: US 2026/0199320 A1
(43) Pub. Date: Jul. 16, 2026

(54) COMBINATION THERAPIES FOR TREATING CANCER

(71) Applicant: INSTITUTE FOR CANCER RESEARCH D/B/A THE RESEARCH INSTITUTE OF FOX CHASE CANCER CENTER, PHILADELPHIA, PA (US)

(72) Inventors: Vladimir KOLENKO, Philadelphia, PA (US); Petr MAKHOV, Philadelphia, PA (US)

(73) Assignee: INSTITUTE FOR CANCER RESEARCH D/B/A THE RESEARCH INSTITUTE OF FOX CHASE CANCER CENTER, PHILADELPHIA, PA (US)

(21) Appl. No.: 19/138,053

(22) PCT Filed: Dec. 13, 2023
(86) PCT No.: PCT/US2023/083858

§ 371 Date: Jun. 11, 2025

Related U.S. Application Data
(60) Provisional application No. 63/432,816, filed on Dec. 15, 2022.

(51) Int. Cl. A61K 31/4709; A61K 31/405; A61K 31/4545; A61K 45/06; A61P 35/00

(52) U.S. Cl. CPC A61K 31/4709; A61K 31/405; A61K 31/4545; A61K 45/06; A61P 35/00

(57) ABSTRACT

Pharmaceutical compositions comprising a combination of: i) cabozantinib or lenvatinib, and ii) tipifarnib, lonafarnib, or NR1, and methods for preventing or treating cancer by administering the same are disclosed herein.

### REFERENCE TO GOVERNMENT GRANTS

This invention was made with government support under CA216173 awarded by the National Institutes of Health. The government has certain rights in the invention.

### FIELD

The present disclosure is directed, in part, to combinations of tyrosine kinase inhibitors and a second agent chosen from a farnesyltransferase inhibitor or a Rheb inhibitor, or pharmaceutically acceptable salts thereof, compositions comprising the same, kits comprising the same, and methods of preventing or treating cancer by administering the same.

### BACKGROUND

Current targeted molecular strategies employing tyrosine kinase inhibitors (TKIs) have resulted in significant gains in overall survival in certain cancer subtypes. Despite the therapeutic progress, however, complete and robust responses have been noted in only a few cases. Furthermore, in some cancer subtypes patients are primarily refractory to treatment with TKIs. Farnesyltransferase (FTase)-dependent proteins regulate endosomal/lysosomal formation.

### SUMMARY

The present disclosure provides pharmaceutical compositions comprising: a tyrosine kinase inhibitor chosen from cabozantinib and lenvatinib, or a pharmaceutically acceptable salt thereof; a second agent chosen from the farnesyltransferase inhibitor tipifarnib or lonafarnib, or the Rheb inhibitor NR1, or a pharmaceutically acceptable salt thereof; and a pharmaceutically acceptable carrier.

The present disclosure also provides methods of treating cancer in a subject in need thereof comprising: administering to the subject a tyrosine kinase inhibitor chosen from cabozantinib and lenvatinib, or a pharmaceutically acceptable salt thereof; and administering to the subject a second agent chosen from the farnesyltransferase inhibitor tipifarnib or lonafarnib, and the Rheb inhibitor NR1, or a pharmaceutically acceptable salt thereof. In some embodiments, the tyrosine kinase inhibitor and the farnesyltransferase inhibitor are present in a single pharmaceutical composition. In some embodiments, the tyrosine kinase inhibitor and the Rheb inhibitor NR1 are present in a single pharmaceutical composition.

The present disclosure also provides combinations of a tyrosine kinase inhibitor chosen from cabozantinib and lenvatinib, or a pharmaceutically acceptable salt thereof, and a second agent chosen from the farnesyltransferase inhibitor tipifarnib or lonafarnib, or the Rheb inhibitor NR1, or a pharmaceutically acceptable salt thereof, for use in the manufacture of a medicament for treating cancer.

The present disclosure also provides uses of a pharmaceutical composition comprising a tyrosine kinase inhibitor chosen from cabozantinib and lenvatinib, or a pharmaceutically acceptable salt thereof, and a second agent chosen from the farnesyltransferase inhibitor tipifarnib or lonafarnib, or the Rheb inhibitor NR1, or a pharmaceutically acceptable salt thereof, for treating cancer.

### BRIEF DESCRIPTION OF THE DRAWINGS

FIG. 1 shows that the combination of tipifarnib and cabozantinib showed synergism, strong synergism, and very strong synergism in various cell lines at 72 hours.

FIG. 2 shows that the combination of tipifarnib and lenvatinib showed various levels of synergism in various cell lines at 72 hours.

FIG. 3 shows that the combination of tipifarnib and cabozantinib showed synergism in various cell lines at 120 hours.

FIG. 4 shows a Western blot and cell viability of 786-0 clear cell renal adenocarcinoma cell line derivatives treated with sunitinib and the effect of UNC-51-like Kinase 3 Kinase Domain (ULK3 KD) shRNA-mediated knockdown on the antitumor activity of sunitinib alone or in combination with Rheb inhibitor, NR1.

FIG. 5 shows TKI stimulation of lysosomal biogenesis in 786-0 cells.

FIG. 6 shows TKI stimulation of lysosomal biogenesis in SK-45 cells.

FIG. 7 shows TKI stimulation of lysosomal biogenesis in RCC4 cells.

FIG. 8 shows tipifarnib reduces lysosomal biogenesis in 786-0 cells.

FIG. 9 shows tipifarnib reduces lysosomal biogenesis in RCC4 cells.

FIG. 10 shows tipifarnib reduces lysosomal biogenesis in 786-O cells.

### DESCRIPTION OF EMBODIMENTS

Various publications, including patents, published applications, technical articles and scholarly articles are cited throughout the specification. Each of these cited publications is incorporated by reference herein, in its entirety and for all purposes.

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the term “animal” includes, but is not limited to, mammals, humans and non-human vertebrates, such as wild, domestic, and farm animals.

As used herein, the term “carrier” means a diluent, adjuvant, or excipient with which a compound is administered in a composition.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the terms “individual,” “subject,” and “patient,” used interchangeably, mean any animal described herein.

As used herein, the phrase “in need thereof” means that the “individual,” “subject,” or “patient” has been identified as having a specific need for the particular method, prevention, or treatment. In some embodiments, the identification can be by any means of diagnosis. In any of the methods, preventions, and treatments described herein, the “individual,” “subject,” or “patient” can be in need thereof.

As used herein, the term “mammal” means a rodent (i.e., a mouse, a rat, or a guinea pig), a monkey, a sheep, a cat, a dog, a cow, a horse, a pig, or a human. In some embodiments, the mammal is a human.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, malic, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, malate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor, or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The present disclosure provides pharmaceutical compositions comprising: i) a tyrosine kinase inhibitor chosen from cabozantinib and lenvatinib, or a pharmaceutically acceptable salt thereof; ii) a second agent chosen from the farnesyltransferase inhibitor tipifarnib or lonafarnib, or the Rheb inhibitor NR1, or a pharmaceutically acceptable salt thereof; and iii) a pharmaceutically acceptable carrier. In some embodiments, the tyrosine kinase inhibitor is cabozantinib, or a pharmaceutically acceptable salt thereof. In some embodiments, the tyrosine kinase inhibitor is lenvatinib. In some embodiments, the second agent is the farnesyltransferase inhibitor tipifarnib, or a pharmaceutically acceptable salt thereof. In some embodiments, the second agent is the farnesyltransferase inhibitor lonafarnib, or a pharmaceutically acceptable salt thereof. In some embodiments, the second agent is the Rheb inhibitor NR1, or a pharmaceutically acceptable salt thereof. In some embodiments, the pharmaceutical composition comprises cabozantinib, or a pharmaceutically acceptable salt thereof, and tipifarnib, or a pharmaceutically acceptable salt thereof. In some embodiments, the pharmaceutical composition comprises cabozantinib, or a pharmaceutically acceptable salt thereof, lonafarnib, or a pharmaceutically acceptable salt thereof. In some embodiments, the pharmaceutical composition comprises cabozantinib, or a pharmaceutically acceptable salt thereof, and NR1, or a pharmaceutically acceptable salt thereof. In some embodiments, the pharmaceutical composition comprises lenvatinib, or a pharmaceutically acceptable salt thereof, tipifarnib, or a pharmaceutically acceptable salt thereof. In some embodiments, the pharmaceutical composition comprises lenvatinib, or a pharmaceutically acceptable salt thereof, and lonafarnib, or a pharmaceutically acceptable salt thereof. In some embodiments, the pharmaceutical composition comprises lenvatinib, or a pharmaceutically acceptable salt thereof, and NR1, or a pharmaceutically acceptable salt thereof.

In any of the embodiments described herein, the cabozantinib can be cabozantinib(S)-malate. In any of the embodiments described herein, the lenvantinib can be lenvantinib methanesulfonate (i.e., lenvantinib mesylate).

In some embodiments, the ratio of the tyrosine kinase inhibitor to the second agent is from about 0.01:1 to about 100:1 (w/w), from about 0.1:1 to about 10:1 (w/w), or from about 1:1 to about 5:1 (w/w). In some embodiments, the ratio of the tyrosine kinase inhibitor to the second agent is from about 0.01:1 to about 100:1 (w/w). In some embodiments, the ratio of the tyrosine kinase inhibitor to the second agent is from about 0.1:1 to about 10:1 (w/w). In some embodiments, the ratio of the tyrosine kinase inhibitor to the second agent is from about 1:1 to about 5:1 (w/w).

In some embodiments, the tyrosine kinase inhibitor is present in an amount from about 1 mg to about 100 mg, from about 5 mg to about 75 mg, from about 10 mg to about 60 mg, or from about 12.5 mg to about 50 mg, and the second agent is present in an amount from about 1 mg to about 500 mg, from about 50 mg to about 400 mg, from about 75 mg to about 300 mg, or from about 100 mg to about 200 mg. In some embodiments, the tyrosine kinase inhibitor is present in an amount from about 1 mg to about 100 mg. In some embodiments, the tyrosine kinase inhibitor is present in an amount from about 5 mg to about 75 mg. In some embodiments, the tyrosine kinase inhibitor is present in an amount from about 10 mg to about 60 mg. In some embodiments, the tyrosine kinase inhibitor is present in an amount from about 12.5 mg to about 50 mg. In some embodiments, the second agent is present in an amount from about 1 mg to about 500 mg. In some embodiments, the second agent is present in an amount from about 50 mg to about 400 mg. In some embodiments, the second agent is present in an amount from about 75 mg to about 300 mg. In some embodiments, the second agent is present in an amount from about 100 mg to about 200 mg.

In some embodiments, the pharmaceutical composition is an oral dosage form, an intravenous dosage form, a topical dosage form, an intraperitoneal dosage form, or an intrathecal dosage form. In some embodiments, the pharmaceutical composition is an oral dosage form or an intravenous dosage form. In some embodiments, the pharmaceutical composition is an oral dosage form.

In some embodiments, the oral dosage form is a pill, tablet, capsule, cachet, gel-cap, pellet, powder, granule, or liquid. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the oral dosage form comprises about 12.5 mg, about 25 mg, about 37.5 mg, or about 50 mg of the tyrosine kinase inhibitor. In some embodiments, the oral dosage form comprises about 12.5 mg of the tyrosine kinase inhibitor. In some embodiments, the oral dosage form comprises about 25 mg of the tyrosine kinase inhibitor. In some embodiments, the oral dosage form comprises about 37.5 mg of the tyrosine kinase inhibitor. In some embodiments, the oral dosage form comprises about 50 mg of the tyrosine kinase inhibitor.

In some embodiments, the oral dosage form comprises about 25 mg, about 50 mg, about 75 mg, about 100 mg, about 150 mg, or about 200 mg of the second agent. In some embodiments, the oral dosage form comprises about 25 mg of the second agent. In some embodiments, the oral dosage form comprises about 50 mg of the second agent. In some embodiments, the oral dosage form comprises about 75 mg of the second agent. In some embodiments, the oral dosage form comprises about 100 mg of the second agent. In some embodiments, the oral dosage form comprises about 150 mg of the second agent. In some embodiments, the oral dosage form comprises about 200 mg of the second agent.

In any of the embodiments described herein, cabozantinib can be administered in the amount of 20 mg, 40 mg, or 60 mg. In any of the embodiments described herein, lenvantinib can be administered in the amount of 4 mg, 8 mg, 10 mg, 12 mg, 18 mg, 20 mg, or 24 mg. In any of the embodiments described herein, lonafarnib can be administered in the amount of 115 mg/m² twice daily or 150 mg/m² twice daily, where the total daily dose is rounded to the nearest 25 mg increment, and 50 mg and 75 mg dosage forms. In any of the embodiments described herein, tipifarnib can be administered in the amount of 100 mg to 1 g daily and dosage forms with 100 mg, 200 mg, or 300 mg.

In some embodiments, the oral dosage form is protected from light and present within a blister pack, bottle, or intravenous bag. In some embodiments, the oral dosage form is present within a blister pack, bottle, or intravenous bag. In some embodiments, the oral dosage form is present within a blister pack. In some embodiments, the oral dosage form is present within a bottle. In some embodiments, the oral dosage form is present within an intravenous bag.

The present disclosure also provides methods of treating cancer in a subject in need thereof comprising: administering to the subject a tyrosine kinase inhibitor chosen from cabozantinib and lenvatinib, or a pharmaceutically acceptable salt thereof; and administering to the subject a second agent chosen from the farnesyltransferase inhibitor tipifarnib or lonafarnib, or the Rheb inhibitor NR1, or a pharmaceutically acceptable salt thereof. In some embodiments, the tyrosine kinase inhibitor is cabozantinib, or a pharmaceutically acceptable salt thereof. In some embodiments, the tyrosine kinase inhibitor is lenvatinib. In some embodiments, the second agent is the farnesyltransferase inhibitor tipifarnib, or a pharmaceutically acceptable salt thereof. In some embodiments, the second agent is the farnesyltransferase inhibitor lonafarnib, or a pharmaceutically acceptable salt thereof. In some embodiments, the second agent is the Rheb inhibitor NR1, or a pharmaceutically acceptable salt thereof.

In some embodiments, the ratio of the administered tyrosine kinase inhibitor to the second agent is from about 0.01:1 to about 100:1 (w/w), from about 0.1:1 to about 10:1 (w/w), or from about 1:1 to about 5:1 (w/w). In some embodiments, the ratio of the administered tyrosine kinase inhibitor to the second agent is from about 0.01:1 to about 100:1 (w/w). In some embodiments, the ratio of the administered tyrosine kinase inhibitor to the second agent is from about 0.1:1 to about 10:1 (w/w). In some embodiments, the ratio of the administered tyrosine kinase inhibitor to the second agent is from about 1:1 to about 5:1 (w/w).

In some embodiments, the tyrosine kinase inhibitor is administered in an amount from about 1 mg to about 100 mg, from about 5 mg to about 75 mg, from about 10 mg to about 60 mg, or from about 12.5 mg to about 50 mg, and the second agent is administered in an amount from about 1 mg to about 500 mg, from about 50 mg to about 400 mg, from about 75 mg to about 300 mg, or from about 100 mg to about 200 mg. In some embodiments, the tyrosine kinase inhibitor is administered in an amount from about 1 mg to about 100 mg. In some embodiments, the tyrosine kinase inhibitor is administered in an amount from about 5 mg to about 75 mg. In some embodiments, the tyrosine kinase inhibitor is administered in an amount from about 10 mg to about 60 mg. In some embodiments, the tyrosine kinase inhibitor is administered in an amount from about 12.5 mg to about 50 mg. In some embodiments, the second agent is administered in an amount from about 1 mg to about 500 mg. In some embodiments, the second agent is administered in an amount from about 50 mg to about 400 mg. In some embodiments, the second agent is administered in an amount from about 75 mg to about 300 mg. In some embodiments, the second agent is administered in an amount from about 100 mg to about 200 mg.

In some embodiments, the tyrosine kinase inhibitor is administered prior to the administration of the second agent or after administration of the second agent. In some embodiments, the tyrosine kinase inhibitor is administered prior to the administration of the second agent. In some embodiments, the tyrosine kinase inhibitor is administered after administration of the second agent. In some embodiments, the tyrosine kinase inhibitor is administered concurrently with administration of the second agent.

In some embodiments, the tyrosine kinase inhibitor and the second agent are administered together in the same pharmaceutical composition. In some embodiments, the pharmaceutical composition comprises cabozantinib, or a pharmaceutically acceptable salt thereof, and tipifarnib, or a pharmaceutically acceptable salt thereof. In some embodiments, the pharmaceutical composition comprises cabozantinib, or a pharmaceutically acceptable salt thereof, lonafarnib, or a pharmaceutically acceptable salt thereof. In some embodiments, the pharmaceutical composition comprises cabozantinib, or a pharmaceutically acceptable salt thereof, and NR1, or a pharmaceutically acceptable salt thereof. In some embodiments, the pharmaceutical composition comprises lenvatinib, or a pharmaceutically acceptable salt thereof, tipifarnib, or a pharmaceutically acceptable salt thereof. In some embodiments, the pharmaceutical composition comprises lenvatinib, or a pharmaceutically acceptable salt thereof, and lonafarnib, or a pharmaceutically acceptable salt thereof. In some embodiments, the pharmaceutical composition comprises lenvatinib, or a pharmaceutically acceptable salt thereof, and NR1, or a pharmaceutically acceptable salt thereof.

In some embodiments, the tyrosine kinase inhibitor is present in a pharmaceutical composition which is an oral dosage form, an intravenous dosage form, a topical dosage form, an intraperitoneal dosage form, or an intrathecal dosage form. In some embodiments, the tyrosine kinase inhibitor is present in a pharmaceutical composition which is an oral dosage form or an intravenous dosage form. In some embodiments, the tyrosine kinase inhibitor is present in a pharmaceutical composition which is an oral dosage form. In some embodiments, the oral dosage form is a pill, tablet, capsule, cachet, gel-cap, pellet, powder, granule, or liquid. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the oral dosage form comprises about 12.5 mg, about 25 mg, about 37.5 mg, or about 50 mg of the tyrosine kinase inhibitor. In some embodiments, the oral dosage form comprises about 12.5 mg of the tyrosine kinase inhibitor. In some embodiments, the oral dosage form comprises about 25 mg of the tyrosine kinase inhibitor. In some embodiments, the oral dosage form comprises about 37.5 mg of the tyrosine kinase inhibitor. In some embodiments, the oral dosage form comprises about 50 mg of the tyrosine kinase inhibitor.

In some embodiments, the second agent is present in a pharmaceutical composition which is an oral dosage form, an intravenous dosage form, a topical dosage form, an intraperitoneal dosage form, or an intrathecal dosage form. In some embodiments, the second agent is present in a pharmaceutical composition which is an oral dosage form or an intravenous dosage form. In some embodiments, the second agent is present in a pharmaceutical composition which is an oral dosage form. In some embodiments, the oral dosage form is a pill, tablet, capsule, cachet, gel-cap, pellet, powder, granule, or liquid. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the oral dosage form comprises about 25 mg, about 50 mg, about 75 mg, about 100 mg, about 150 mg, or about 200 mg of the second agent. In some embodiments, the oral dosage form comprises about 25 mg of the second agent. In some embodiments, the oral dosage form comprises about 50 mg of the second agent. In some embodiments, the oral dosage form comprises about 75 mg of the second agent. In some embodiments, the oral dosage form comprises about 100 mg of the second agent. In some embodiments, the oral dosage form comprises about 150 mg of the second agent. In some embodiments, the oral dosage form comprises about 200 mg of the second agent.

In some embodiments, the tyrosine kinase inhibitor and the second agent are administered to the subject together in a single pharmaceutical composition. In some embodiments, the single pharmaceutical composition is an oral dosage form, an intravenous dosage form, a topical dosage form, an intraperitoneal dosage form, or an intrathecal dosage form. In some embodiments, the single pharmaceutical composition is an oral dosage form or an intravenous dosage form. In some embodiments, the single pharmaceutical composition is an oral dosage form. In some embodiments, the single pharmaceutical composition is an intravenous dosage form. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the single pharmaceutical composition comprises about 12.5 mg, about 25 mg, about 37.5 mg, or about 50 mg of the tyrosine kinase inhibitor. In some embodiments, the single pharmaceutical composition comprises about 12.5 mg of the tyrosine kinase inhibitor. In some embodiments, the single pharmaceutical composition comprises about 25 mg of the tyrosine kinase inhibitor. In some embodiments, the single pharmaceutical composition comprises about 37.5 mg of the tyrosine kinase inhibitor. In some embodiments, the single pharmaceutical composition comprises about 50 mg of the tyrosine kinase inhibitor.

In some embodiments, the single pharmaceutical composition comprises about 25 mg, about 50 mg, about 75 mg, about 100 mg, about 150 mg, or about 200 mg of the second agent. In some embodiments, the single pharmaceutical composition comprises about 25 mg of the second agent. In some embodiments, the single pharmaceutical composition comprises about 50 mg of the second agent. In some embodiments, the single pharmaceutical composition comprises about 75 mg of the second agent. In some embodiments, the single pharmaceutical composition comprises about 100 mg of the second agent. In some embodiments, the single pharmaceutical composition comprises about 150 mg of the second agent. In some embodiments, the single pharmaceutical composition comprises about 200 mg of the second agent.

In some embodiments, the subject is also administered radiation therapy, immunotherapy, and/or neoadjuvant therapy. In some embodiments, the subject is also administered radiation therapy. In some embodiments, the subject is also administered immunotherapy. In some embodiments, the subject is also administered neoadjuvant therapy.

In some embodiments, the cancer is breast cancer, pancreatic cancer, prostate cancer, lung cancer, liver cancer, esophageal cancer, stomach cancer, billary tract cancer, head and neck cancer, bladder cancer, kidney cancer, mesothelioma, thyroid cancer, uterine cancer, ovarian cancer, brain cancer, lymphoma, myeloma, leukemia, or colon cancer. In some embodiments, the cancer is kidney cancer. In some embodiments, the cancer is renal cell carcinoma, clear cell renal carcinoma (ccRCC), papillary renal cell carcinoma, chromophobe renal cell carcinoma, or unclassified renal cell carcinoma. In some embodiments, the cancer is renal cell carcinoma. In some embodiments, the cancer is clear cell renal carcinoma (ccRCC). In some embodiments, the cancer is papillary renal cell carcinoma. In some embodiments, the cancer is chromophobe renal cell carcinoma. In some embodiments, the cancer is unclassified renal cell carcinoma.

In some embodiments, the cancer is a TKI-resistant cancer or a TKI-refractory cancer. In some embodiments, the cancer is a refractory or intolerant gastrointestinal stromal tumor, or advanced renal cell carcinoma, or metastatic castration-refractory prostate cancer.

The compounds and compositions described herein can be administered by any route of administration including, but not limited to, oral, intravenous, topical, intraperitoneal, and intrathecal. In some embodiments, the administration is oral, intravenous, intraperitoneal, or intrathecal. In some embodiments, the administration is oral, intravenous, or intraperitoneal. In some embodiments, the administration is oral or intravenous. In some embodiments, the administration is oral or topical. In some embodiments, the administration is oral or intraperitoneal. In some embodiments, the administration is oral or intrathecal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or composition(s) comprising the same to a particular area in need of treatment. This may be achieved, for example, by local infusion (for example, during surgery), topical application (for example, with a wound dressing after surgery), or by injection (for example, by depot injection). Formulations for injection can be presented in unit dosage form, such as in ampoules or in multi-dose containers, with an added preservative.

The compounds and compositions described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a subject to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compounds and compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The compositions described herein can be administered either alone (as a single composition comprising the compounds described herein) or in combination (concurrently or serially) with other pharmaceutical agents. For example, the compounds and compositions can be administered in combination with anti-cancer or anti-neoplastic agents (for example, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel) or therapies (for example, surgery or radiotherapy).

The amount of any particular compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Suitable compositions include, but are not limited to, oral non-absorbed compositions. Suitable compositions also include, but are not limited to saline, water, cyclodextrin solutions, and buffered solutions of pH 3-9.

The compounds and compositions described herein can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the compounds and compositions described herein can be lyophilized to a solid and reconstituted with, for example, water prior to use.

When administered to a human, the compounds and compositions can be sterile. Water is a suitable carrier when the compound and composition is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds and compositions are formulated in accordance with routine procedures as pharmaceutical compositions adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound or composition is to be administered by infusion, it can be dispensed, for example, with an infusion bottle containing sterile pharmaceutical grade water or saline. Where the compound or composition is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present invention is in the form of a liquid wherein the active agents are present in solution, in suspension, as an emulsion, or as a solution/suspension. In some embodiments, the liquid composition is in the form of a gel. In other embodiments, the liquid composition is aqueous. In other embodiments, the composition is in the form of an ointment.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

The present disclosure also provides combinations of a tyrosine kinase inhibitor chosen from cabozantinib and lenvatinib, or a pharmaceutically acceptable salt thereof, and a second agent chosen from the farnesyltransferase inhibitor tipifarnib or lonafarnib, or the Rheb inhibitor NR1, or a pharmaceutically acceptable salt thereof, for use in the manufacture of a medicament for treating cancer. Any of the combinations described herein can be used in the manufacture of a medicament for treating any of the cancers described herein.

The present disclosure also provides uses of a pharmaceutical composition comprising a tyrosine kinase inhibitor chosen from cabozantinib and lenvatinib, or a pharmaceutically acceptable salt thereof, and a second agent chosen from the farnesyltransferase inhibitor tipifarnib or lonafarnib, or the Rheb inhibitor NR1, or a pharmaceutically acceptable salt thereof, for treating cancer. Any of the combinations described herein can be used for treating any of the cancers described herein.

The present disclosure also provides methods of treating or reducing a cancer, inhibiting tumor growth, or treating or preventing spread or metastasis of cancer (e.g., any of the cancers described herein) in a mammal comprising administering to the mammal in need thereof any of the combinations of compounds, or compositions comprising the same as described herein. In some embodiments, one or more compounds may be combined in the same composition for any of the methods disclosed herein.

The present disclosure also provides methods for killing or inhibiting growth of a cancer cell comprising contacting the cancer cell with any of the combinations of compounds, or compositions comprising the same as described herein. In some embodiments, one or more compounds may be combined in the same composition for any of the methods disclosed herein.

Thus, the compounds and compositions can be used as anti-cancer and anti-tumor agents, e.g., the compounds can kill or inhibit the growth of cancer cells. The compounds and compositions can also be used in methods of reducing cancer in an animal, or in methods of treating or preventing the spread or metastasis of cancer in an animal, or in methods of treating an animal afflicted with cancer. The compounds and compositions can also be used in methods of killing or inhibiting the growth of a cancer cell, or in methods of inhibiting tumor growth.

Generally, cancer refers to any malignant growth or tumor caused by abnormal and uncontrolled cell division; it may spread to other parts of the body through the lymphatic system or the blood stream. Cancers include both solid tumors and blood-borne tumors.

Cancers that are treatable are broadly divided into the categories of carcinoma, lymphoma and sarcoma. Examples of carcinomas include, but are not limited to: adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Sarcomas include, but are not limited to: amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lympho sarcoma), medullary sarcoma, myeloid sarcoma (granulocitic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Lymphomas include, but are not limited to: Hodgkin's disease and lymphocytic lymphomas, such as Burkitt's lymphoma, NPDL, NML, NH and diffuse lymphomas.

Examples of cancers that can be treated using the compounds described herein include, but are not limited to, Hodgkin's disease, non-Hodgkin's lymphomas, acute lymphocytic leukemia, multiple myeloma, breast carcinomas, ovarian carcinomas, lung carcinomas, Wilms' tumor, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, rhabdomyosarcoma, Kaposi's sarcoma, genitourinary carcinomas, thyroid carcinomas, esophageal carcinomas, malignant hypercalcemia, renal cell carcinomas, endometrial carcinomas, polycythemia vera, essential thrombocytosis, adrenal cortex carcinomas, skin cancer, and prostatic carcinomas.

In some embodiments, the cancer is lung cancer (such as non-small cell lung cancer), breast cancer, prostate cancer, ovarian cancer, testicular cancer, colon cancer, renal cancer, bladder cancer, pancreatic cancer, glioblastoma, neuroblastoma, sarcomas such as Kaposi's sarcoma and Ewing's sarcoma, hemangiomas, solid tumors, blood-borne tumors, rhabdomyosarcoma, CNS cancer (such as brain cancer), retinoblastoma, neuroblastoma, leukemia, melanoma, kidney or renal cancer, and osteosarcoma.

The compounds and compositions can be used in methods of killing or inhibiting the growth of cancer cells, either in vivo or in vitro, or inhibiting the growth of a cancerous tumor.

In some embodiments, the compounds and compositions are used in conjunction with other therapies, such as standard immunotherapy, neoadjuvant therapy, radiotherapy, tumor surgery, and conventional chemotherapy directed against solid tumors and for the control of establishment of metastases. Additionally, the compounds and compositions can be administered after surgery where solid tumors have been removed as a prophylaxis against metastasis. Cytotoxic or chemotherapeutic agents include, but are not limited to, aziridine thiotepa, alkyl sulfonate, nitrosoureas, platinum complexes, NO classic alkylators, folate analogs, purine analogs, adenosine analogs, pyrimidine analogs, substituted urea, antitumor antibiotics, microtubule agents, and asparaginase.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: The Combined Treatment with TKIs and Tipifarnib on the Viability of ccRCC Cells Shows Synergy

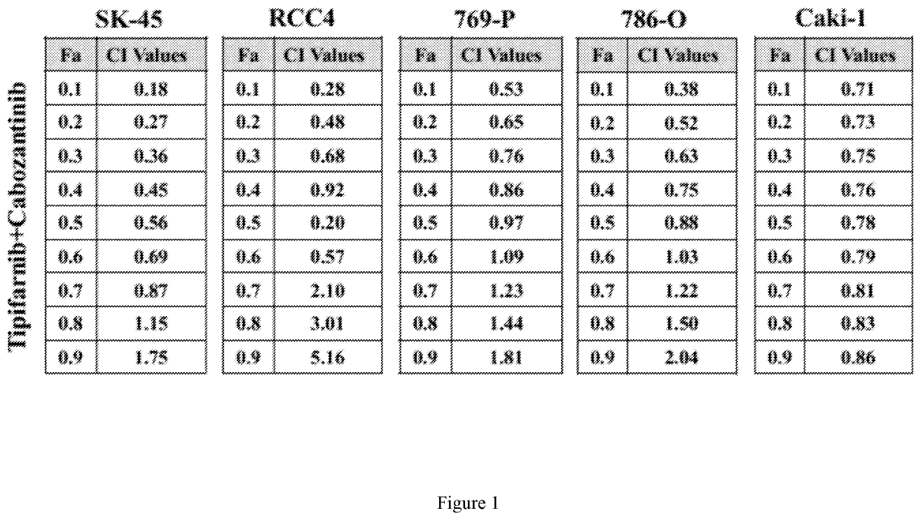

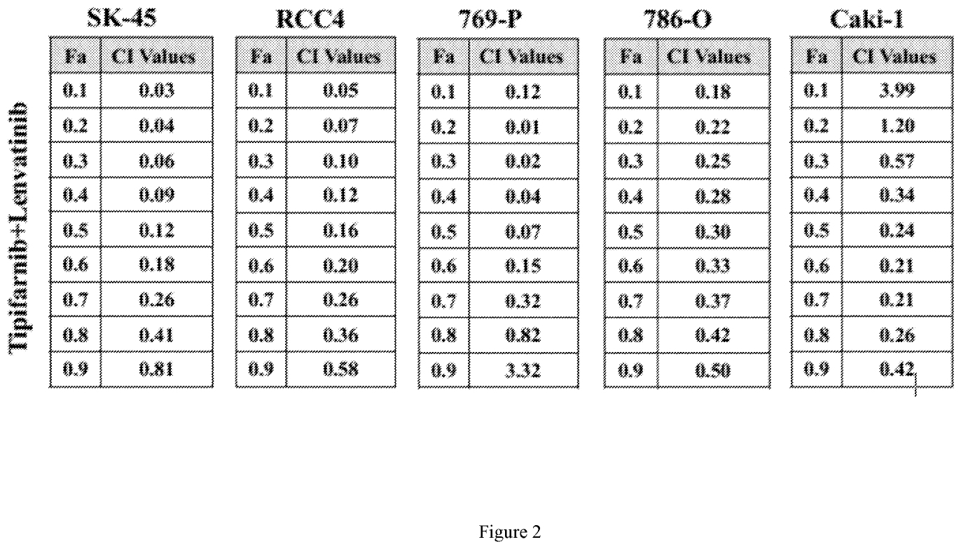

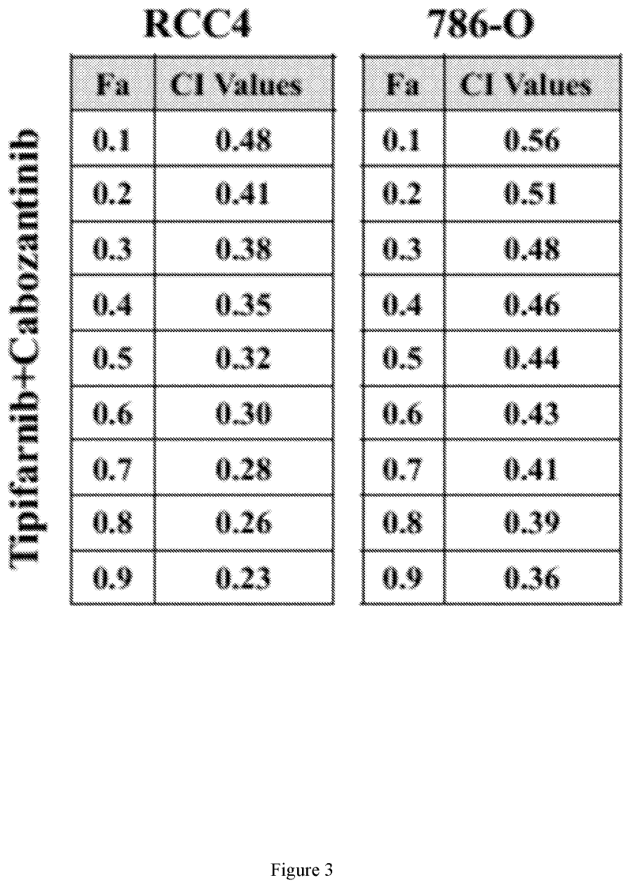

The synergistic effect of combined treatment with TKIs and tipifarnib on the viability of ccRCC cells is demonstrated in FIG. 1, FIG. 2, and FIG. 3. ccRCC cells were treated with escalating concentrations of TKIs and tipifarnib for 72 hours. Cell viability was analyzed by CellTiter Blue assay (Promega). The data analysis was performed using CalcuSyn 2.0 software. Fa, effect value. CI, combination index. The cell line 786-O is a sunitinib-resistant renal cell carcinoma cell line.

The Combination Index (CI) values disclosed in FIG. 1, FIG. 2, and FIG. 3 are:
0.85 to 0.9=slight synergism;
0.7 to 0.85=moderate synergism;
0.3 to 0.7=synergism;
0.1 to 0.3=strong synergism; and
<0.1=very strong synergism.

As can be seen in FIG. 1, the combination of tipifarnib and cabozantinib showed synergism, strong synergism, and very strong synergism in various cell lines at 72 hours. As can be seen in FIG. 2, the combination of tipifarnib and lenvatinib showed various levels of synergism in various cell lines at 72 hours. As can be seen in FIG. 3, the combination of tipifarnib and cabozantinib showed synergism in various cell lines at 120 hours.

### Example 2: Lonafarnib/Sorafenib Combination Treatment of HEP 3B Hepatocellular Carcinoma

Cell viability was analyzed by CellTiter Blue assay. ED, effective dose. The data analysis was performed using CalcuSyn 2.0 software. Combination index (CI) was calculated as described previously (Chou, Pharmacol. Rev. 2006, 58, 621-81). CI 0.85-0.9: slight synergism; CI 0.7-0.85: moderate synergism; CI 0.3-0.7: synergism; CI 0.1-0.3: strong synergism; CI<0.1: very strong synergism.

CI Data for Drug Combo: LonSor (Lon+Sor [100:1])

| Fa | CI Value | Total Dose |
|---|---|---|
| 0.05 | 0.25448 | 1.37025 |
| 0.1 | 0.23856 | 4.13492 |
| 0.15 | 0.23631 | 8.19304 |
| 0.2 | 0.23844 | 13.7100 |
| 0.25 | 0.24271 | 20.9755 |
| 0.3 | 0.24833 | 30.4118 |
| 0.35 | 0.25502 | 42.6159 |
| 0.4 | 0.26268 | 58.4351 |
| 0.45 | 0.27135 | 79.0933 |
| 0.5 | 0.28113 | 106.404 |
| 0.55 | 0.29225 | 143.146 |
| 0.6 | 0.30503 | 193.751 |
| 0.65 | 0.31997 | 265.672 |
| 0.7 | 0.33786 | 372.285 |
| 0.75 | 0.35998 | 539.765 |
| 0.8 | 0.38864 | 825.809 |
| 0.85 | 0.42852 | 1381.89 |
| 0.9 | 0.49123 | 2738.11 |
| 0.95 | 0.62006 | 8262.64 |
| 0.97 | 0.73661 | 18130.7 |

CI values for actual experimental points:

| Total Dose | Fa | CI Value |
|---|---|---|
| 1.01E−4 | 1.0E−4 | 1.08591 |
| 101.0 | 0.34468 | 0.62388 |
| 202.0 | 0.37774 | 1.02890 |
| 404.0 | 0.60837 | 0.60858 |
| 606.0 | 0.84428 | 0.19807 |
| 808.0 | 0.93635 | 0.0816] |

### Example 3: Lonafarnib/Lenvatinib Combination Treatment of HEP 3B Hepatocellular Carcinoma

Cell viability was analyzed by CellTiter Blue assay. ED, effective dose. The data analysis was performed using CalcuSyn 2.0 software. Combination index (CI) was calculated as described previously (Chou, Pharmacol. Rev. 2006, 58, 621-81). CI 0.85-0.9: slight synergism; CI 0.7-0.85: moderate synergism; CI 0.3-0.7: synergism; CI 0.1-0.3: strong synergism; CI<0.1: very strong synergism.

CI Data for Drug Combo: LonLen (Lon+Len [100:1])

| Fa | CI Value | Total Dose |
|---|---|---|
| 0.05 | 0.26337 | 1.05399 |
| 0.1 | 0.27627 | 3.16664 |
| 0.15 | 0.29478 | 6.25741 |
| 0.2 | 0.31454 | 10.4496 |
| 0.25 | 0.33496 | 15.9602 |
| 0.3 | 0.35607 | 23.1061 |
| 0.35 | 0.37808 | 32.3350 |
| 0.4 | 0.40129 | 44.2822 |
| 0.45 | 0.42608 | 59.8649 |
| 0.5 | 0.45295 | 80.4413 |
| 0.55 | 0.48253 | 108.090 |
| 0.6 | 0.51573 | 146.126 |
| 0.65 | 0.55381 | 200.118 |
| 0.7 | 0.59873 | 280.048 |
| 0.75 | 0.65366 | 405.433 |
| 0.8 | 0.72425 | 619.241 |
| 0.85 | 0.82202 | 1034.10 |
| 0.9 | 0.97576 | 2043.43 |
| 0.95 | 1.29423 | 6139.32 |
| 0.97 | 1.58674 | 13429.4 |

### Example 4: Treatment of HEP 3B Hepatocellular Carcinoma

Cell viability was analyzed by CellTiter Blue assay. ED, effective dose. The data analysis was performed using CalcuSyn 2.0 software. Combination index (CI) was calculated as described previously (Chou, Pharmacol. Rev. 2006, 58, 621-81). CI 0.85-0.9: slight synergism; CI 0.7-0.85: moderate synergism; CI 0.3-0.7: synergism; CI 0.1-0.3: strong synergism; CI<0.1: very strong synergism. The following treatments were carried out: Lonafarnib (Lon) [nM], Sorafenib (Sor) [uM], Lenvatinib (Len) [uM], Lonafarnib+Sorafenib (LonSor) (Lon+Sor [100:1]), and Lonafarnib+Lenvatinib (LonLen) (Lon+Len [100:1]).

Data for Drug: Lon [nM]

| Dose | Effect |
|---|---|
| 1.0E−4 | 1.0E−4 |
| 100.0 | 0.21707 |
| 200.0 | 0.27916 |
| 400.0 | 0.31243 |
| 600.0 | 0.32473 |
| 800.0 | 0.37466 |

| | |
|---|---|
| X-int: | 3.19263 |
| Y-int: | −1.7674 +/− 0.04066 |
| m: | 0.55357 +/− 0.01434 |
| Dm: | 1558.23 |
| r: | 0.99866 |

Data for Drug: Sor [uM]

| Dose | Effect |
|---|---|
| 1.0E−4 | 1.0E−4 |
| 1.0 | 0.05824 |
| 2.0 | 0.1667 |
| 4.0 | 0.4078 |
| 6.0 | 0.67623 |
| 8.0 | 0.86806 |

| | |
|---|---|
| X-int: | 0.69320 |
| Y-int: | −0.6132 +/− 0.20274 |
| m: | 0.88460 +/− 0.11746 |
| Dm: | 4.93396 |
| r: | 0.96650 |

Data for Drug: Len [uM]

| Dose | Effect |
|---|---|
| 1.0E−4 | 1.0E−4 |
| 1.0 | 0.34871 |
| 2.0 | 0.59084 |
| 4.0 | 0.68353 |
| 6.0 | 0.70605 |
| 8.0 | 0.73159 |

| | |
|---|---|
| X-int: | 0.29711 |
| Y-int: | −0.2751 +/− 0.04490 |
| m: | 0.92587 +/− 0.02601 |
| Dm: | 1.98203 |
| r: | 0.99842 |

Data for Drug Combo: LonSor (Lon+Sor [100:1])

| Dose A | Effect |
|---|---|
| 1.0E−4+ | 1.0E−4 |
| 100.0+ | 0.34468 |
| 200.0+ | 0.37774 |
| 400.0+ | 0.60837 |
| 600.0+ | 0.84428 |
| 800.0+ | 0.93635 |

| | |
|---|---|
| X-int: | 2.02696 |
| Y-int: | −1.3713 +/− 0.19172 |
| m: | 0.67653 +/− 0.06758 |
| Dm: | 106.404 |
| r: | 0.98062 |

Data for Drug Combo: LonLen (Lon+Len [100:1])

| Dose A | Effect |
|---|---|
| 1.0E−4+ | 1.0E−4 |
| 100.0+ | 0.48908 |
| 200.0+ | 0.66115 |
| 400.0+ | 0.76439 |
| 600.0+ | 0.79346 |
| 800.0+ | 0.83864 |

| | |
|---|---|
| X-int: | 1.90548 |
| Y-int: | −1.2943 +/− 0.02416 |
| m: | 0.67923 +/− 0.00852 |
| Dm: | 80.4413 |
| r: | 0.99969 |

CI Data for Drug Combo: LonSor (Lon+Sor [100:1])

| Fa | CI Value | Total Dose |
|---|---|---|
| 0.05 | 0.25448 | 1.37025 |
| 0.1 | 0.23856 | 4.13492 |
| 0.15 | 0.23631 | 8.19304 |
| 0.2 | 0.23844 | 13.7100 |
| 0.25 | 0.24271 | 20.9755 |
| 0.3 | 0.24833 | 30.4118 |
| 0.35 | 0.25502 | 42.6159 |
| 0.4 | 0.26268 | 58.4351 |
| 0.45 | 0.27135 | 79.0933 |
| 0.5 | 0.28113 | 106.404 |
| 0.55 | 0.29225 | 143.146 |
| 0.6 | 0.30503 | 193.751 |
| 0.65 | 0.31997 | 265.672 |
| 0.7 | 0.33786 | 372.285 |
| 0.75 | 0.35998 | 539.765 |
| 0.8 | 0.38864 | 825.809 |
| 0.85 | 0.42852 | 1381.89 |
| 0.9 | 0.49123 | 2738.11 |
| 0.95 | 0.62006 | 8262.64 |
| 0.97 | 0.73661 | 18130.7 |

| Total Dose | Fa | CI Value |
|---|---|---|
| 1.01E−4 | 1.0E−4 | 1.08591 |
| 101.0 | 0.34468 | 0.62388 |
| 202.0 | 0.37774 | 1.02890 |
| 404.0 | 0.60837 | 0.60858 |
| 606.0 | 0.84428 | 0.19807 |
| 808.0 | 0.93635 | 0.08161 |

CI Data for Drug Combo: LonLen (Lon+Len [100:1])

| Fa | CI Value | Total Dose |
|---|---|---|
| 0.05 | 0.26337 | 1.05399 |
| 0.1 | 0.27627 | 3.16664 |
| 0.15 | 0.29478 | 6.25741 |
| 0.2 | 0.31454 | 10.4496 |
| 0.25 | 0.33496 | 15.9602 |
| 0.3 | 0.35607 | 23.1061 |
| 0.35 | 0.37808 | 32.3350 |
| 0.4 | 0.40129 | 44.2822 |
| 0.45 | 0.42608 | 59.8649 |
| 0.5 | 0.45295 | 80.4413 |
| 0.55 | 0.48253 | 108.090 |
| 0.6 | 0.51573 | 146.126 |
| 0.65 | 0.55381 | 200.118 |
| 0.7 | 0.59873 | 280.048 |
| 0.75 | 0.65366 | 405.433 |
| 0.8 | 0.72425 | 619.241 |
| 0.85 | 0.82202 | 1034.10 |
| 0.9 | 0.97576 | 2043.43 |
| 0.95 | 1.29423 | 6139.32 |
| 0.97 | 1.58674 | 13429.4 |

| Total Dose | Fa | CI Value |
|---|---|---|
| 1.01E−4 | 1.0E−4 | 1.08971 |
| 101.0 | 0.48908 | 0.59836 |
| 202.0 | 0.66115 | 0.52859 |
| 404.0 | 0.76439 | 0.59675 |
| 606.0 | 0.79346 | 0.74135 |
| 808.0 | 0.83864 | 0.70675 |

DRI Data for Drug Combo: LonSor (Lon+Sor [100:1])

| Fa | Dose Lon | Dose Sor | DRI Lon | DRI Sor |
|---|---|---|---|---|
| 0.05 | 7.63181 | 0.17686 | 5.62535 | 13.0363 |
| 0.1 | 29.4334 | 0.41160 | 7.18943 | 10.0537 |
| 0.15 | 67.8864 | 0.69438 | 8.36873 | 8.56000 |
| 0.2 | 127.362 | 1.02943 | 9.38262 | 7.58370 |
| 0.25 | 214.159 | 1.42506 | 10.3120 | 6.86187 |
| 0.3 | 337.210 | 1.89329 | 11.1990 | 6.28775 |
| 0.35 | 509.303 | 2.45064 | 12.0705 | 5.80804 |
| 0.4 | 749.086 | 3.11985 | 12.9473 | 5.39239 |
| 0.45 | 1084.42 | 3.93257 | 13.8478 | 5.02179 |
| 0.5 | 1558.23 | 4.93396 | 14.7909 | 4.68337 |
| 0.55 | 2239.05 | 6.19034 | 15.7982 | 4.36775 |
| 0.6 | 3241.39 | 7.80293 | 16.8970 | 4.06757 |
| 0.65 | 4767.46 | 9.93371 | 18.1243 | 3.77647 |
| 0.7 | 7200.51 | 12.8580 | 19.5348 | 3.48835 |
| 0.75 | 11337.8 | 17.0827 | 21.2150 | 3.19649 |
| 0.8 | 19064.4 | 23.6479 | 23.3165 | 2.89224 |
| 0.85 | 35766.8 | 35.0585 | 26.1414 | 2.56237 |
| 0.9 | 82494.1 | 59.1449 | 30.4294 | 2.18167 |
| 0.95 | 318153. | 137.645 | 38.8900 | 1.68253 |
| 0.97 | 831265. | 251.060 | 46.3070 | 1.39857 |
| 1.0E−4 | 9.27E−5 | 1.48E−4 | 0.92664 | 148.406 |
| 0.34468 | 488.163 | 2.38648 | 4.88163 | 2.38648 |
| 0.37774 | 632.458 | 2.80633 | 3.16229 | 1.40316 |
| 0.60837 | 3452.95 | 8.11785 | 8.63238 | 2.02946 |
| 0.84428 | 33023.4 | 33.3507 | 55.0391 | 5.55846 |
| 0.93635 | 200408. | 103.075 | 250.509 | 12.8843 |

DRI Data for Drug Combo: LonLen (Lon+Len [100:1])

| Fa | Dose Lon | Dose Len | DRI Lon | DRI Len |
|---|---|---|---|---|
| 0.05 | 7.63181 | 0.08241 | 7.31326 | 7.89689 |
| 0.1 | 29.4334 | 0.18470 | 9.38779 | 5.89100 |
| 0.15 | 67.8864 | 0.30442 | 10.9575 | 4.91353 |
| 0.2 | 127.362 | 0.44345 | 12.3102 | 4.28617 |
| 0.25 | 214.159 | 0.60504 | 13.5525 | 3.82887 |
| 0.3 | 337.210 | 0.79373 | 14.7399 | 3.46949 |
| 0.35 | 509.303 | 1.01564 | 15.9084 | 3.17240 |
| 0.4 | 749.086 | 1.27914 | 17.0853 | 2.91750 |
| 0.45 | 1084.42 | 1.59581 | 18.2957 | 2.69234 |
| 0.5 | 1558.23 | 1.98203 | 19.5647 | 2.48858 |
| 0.55 | 2239.05 | 2.46171 | 20.9218 | 2.30024 |
| 0.6 | 3241.39 | 3.07114 | 22.4039 | 2.12272 |
| 0.65 | 4767.46 | 3.86794 | 24.0615 | 1.95216 |
| 0.7 | 7200.51 | 4.94935 | 25.9689 | 1.78500 |
| 0.75 | 11337.8 | 6.49278 | 28.2442 | 1.61746 |
| 0.8 | 19064.4 | 8.85876 | 31.0945 | 1.44489 |
| 0.85 | 35766.8 | 12.9048 | 34.9332 | 1.26040 |
| 0.9 | 82494.1 | 21.2693 | 40.7741 | 1.05127 |
| 0.95 | 318153. | 47.6701 | 52.3404 | 0.78424 |
| 0.97 | 831265. | 84.6505 | 62.5177 | 0.63664 |
| 1.0E−4 | 9.27E−5 | 9.48E−5 | 0.92664 | 94.8194 |
| 0.48908 | 1439.98 | 1.89068 | 14.3998 | 1.89068 |
| 0.66115 | 5212.31 | 4.07985 | 26.0616 | 2.03992 |
| 0.76439 | 13060.1 | 7.06567 | 32.6502 | 1.76642 |
| 0.79346 | 17723.1 | 8.48066 | 29.5385 | 1.41344 |
| 0.83864 | 30595.1 | 11.7543 | 38.2439 | 1.46929 |

### Summary Tables

| Drug/Combo | Dm | m | r |
|---|---|---|---|
| Lon | 1558.23 | 0.55357 | 0.99866 |
| Sor | 4.93396 | 0.88460 | 0.96650 |
| Len | 1.98203 | 0.92587 | 0.99842 |
| LonSor | 106.404 | 0.67653 | 0.98062 |
| LonLen | 80.4413 | 0.67923 | 0.99969 |

CI values at:

| Combo | ED50 | ED75 | ED90 | ED95 |
|---|---|---|---|---|
| LonSor | 0.28113 | 0.35998 | 0.49123 | 0.62006 |
| LonLen | 0.45295 | 0.65366 | 0.97576 | 1.29423 |

Data for Fa=0.1

| Drug/Combo | CI value | Dose Lon | Dose Sor | Dose Len |
|---|---|---|---|---|
| Lon | | 29.4334 | | |
| Sor | | | 0.41160 | |
| Len | | | | 0.18470 |
| LonSor | 0.23856 | 4.09398 | 0.04094 | |
| LonLen | 0.27627 | 3.13528 | | 0.03135 |

Data for Fa=0.25

| Drug/Combo | CI value | Dose Lon | Dose Sor | Dose Len |
|---|---|---|---|---|
| Lon | | 214.159 | | |
| Sor | | | 1.42506 | |
| Len | | | | 0.60504 |
| LonSor | 0.24271 | 20.7679 | 0.20768 | |
| LonLen | 0.33496 | 15.8022 | | 0.15802 |

Data for Fa=0.5

| Drug/Combo | CI value | Dose Lon | Dose Sor | Dose Len |
|---|---|---|---|---|
| Lon | | 1558.23 | | |
| Sor | | | 4.93396 | |
| Len | | | | 1.98203 |
| LonSor | 0.28113 | 105.351 | 1.05351 | |
| LonLen | 0.45295 | 79.6448 | | 0.79645 |

Data for Fa=0.75

| Drug/Combo | CI value | Dose Lon | Dose Sor | Dose Len |
|---|---|---|---|---|
| Lon | | 11337.8 | | |
| Sor | | | 17.0827 | |
| Len | | | | 6.49278 |
| LonSor | 0.35998 | 534.421 | 5.34421 | |
| LonLen | 0.65366 | 401.419 | | 4.01419 |

Data for Fa=0.95

| Drug/Combo | CI value | Dose Lon | Dose Sor | Dose Len |
|---|---|---|---|---|
| Lon | | 318153. | | |
| Sor | | | 137.645 | |
| Len | | | | 47.6701 |
| LonSor | 0.62006 | 8180.83 | 81.8083 | |
| LonLen | 1.29423 | 6078.53 | | 60.7853 |

### Example 5: Effect of ULK3 KD on the Antitumor Activity of Sunitinib

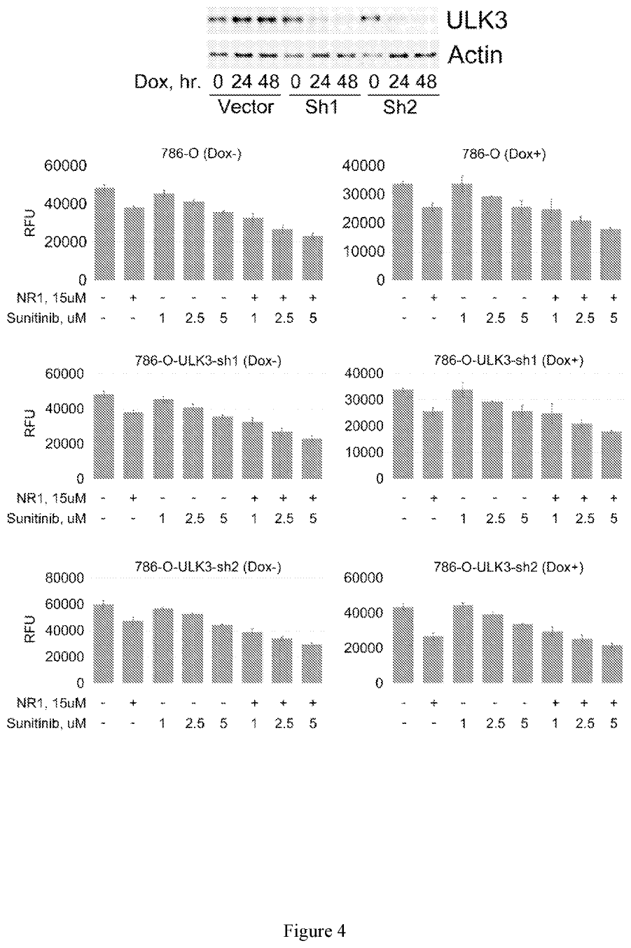

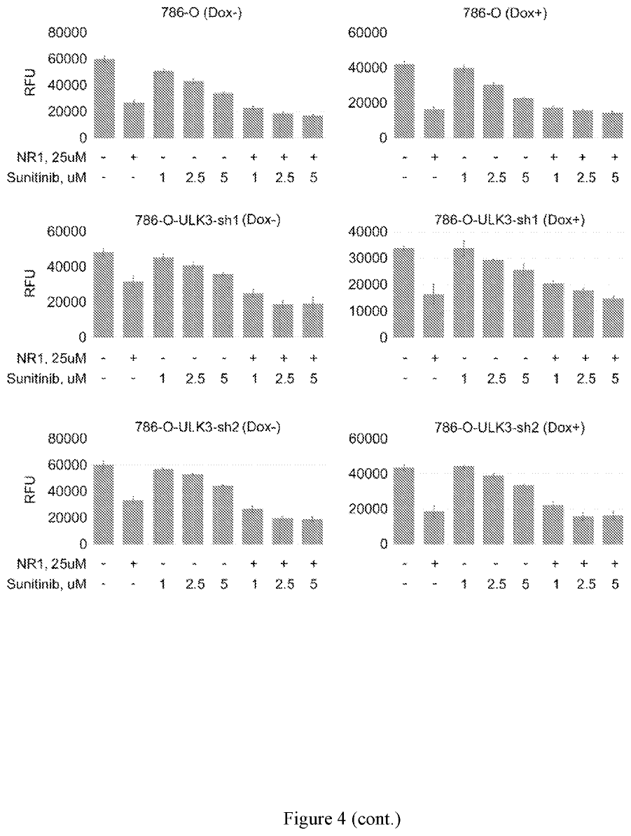

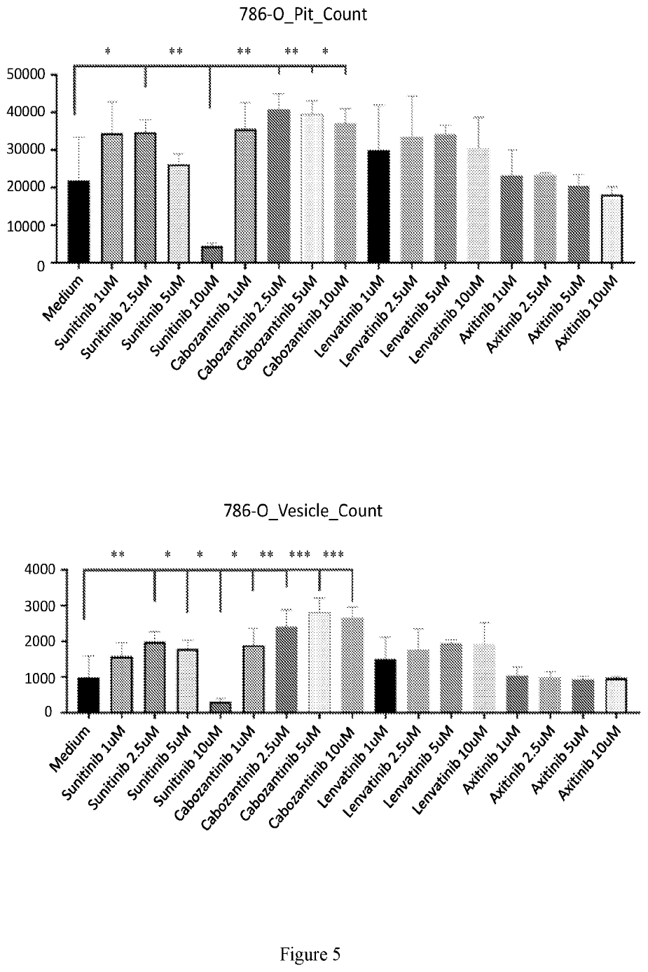

Referring to FIG. 4, the cell viability of 786-0 clear cell renal adenocarcinoma cell line derivatives treated with the indicated amounts of NR1 and/or sunitinib was analyzed by CellTiter Blue assay (Promega). RFU means relative fluorescence units. For Western Blot analysis of the efficacy of ULK3 knockdown, protein concentrations in whole cell lysates were measured with BCA protein assay reagents (Pierce, Rockford, IL). Equivalent amounts of proteins (20 μg) were mixed with an equal volume of 2X Laemmli sample buffer, boiled and resolved by electrophoresis in 10% sodium dodecyl sulfate-polyacrylamide gels (SDS-PAGE). The proteins were transferred from the gel to a PVDF membrane using an electroblotting apparatus (Bio-Rad). Membranes were then incubated in a blocking solution containing 5% nonfat dry milk overnight to inhibit nonspecific binding. The membranes were then incubated with specific antibody 2 hours at room temperature or overnight at 4C. After washing in TBS/0.1% Tween 20 for 30 minutes, membranes were incubated for another 30 minutes with horseradish peroxidase-conjugated secondary antibody. The membranes were then washed and developed with enhanced chemiluminescence (ECL Western Blotting Kit, Amersham, Arlington Heights, IL). The results indicate that NR1 reduces cell viability of sunitinib-treated primary clear cell adenocarcinoma cell lines.

### Example 6: Lysosomal Biogenesis Analysis

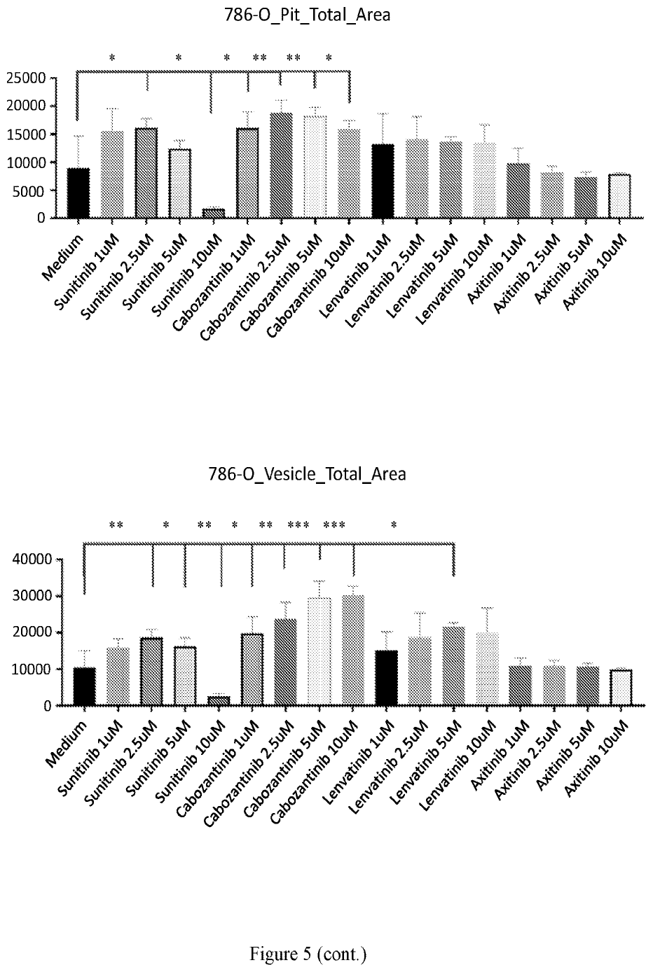

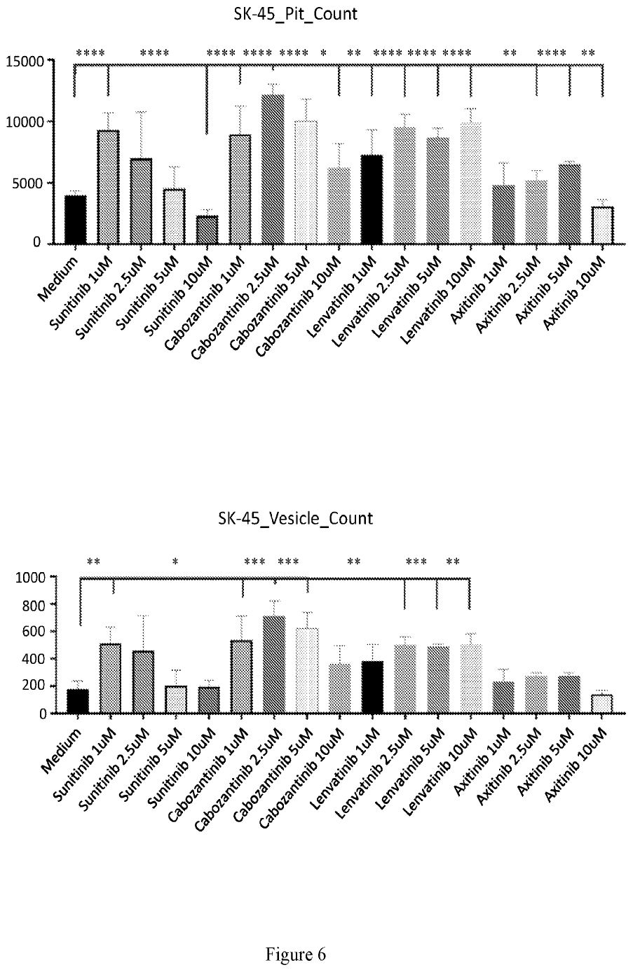

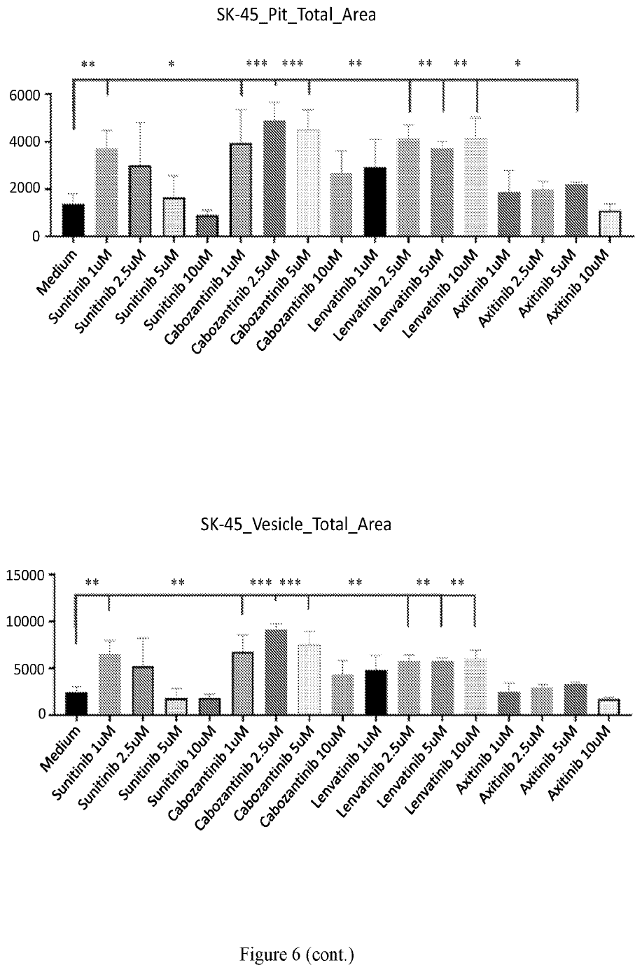

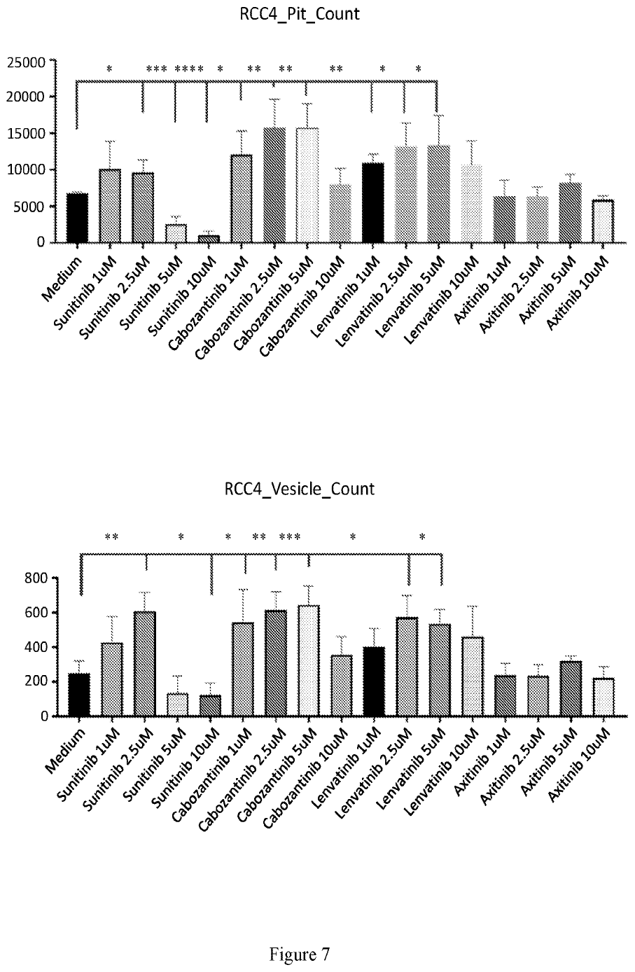

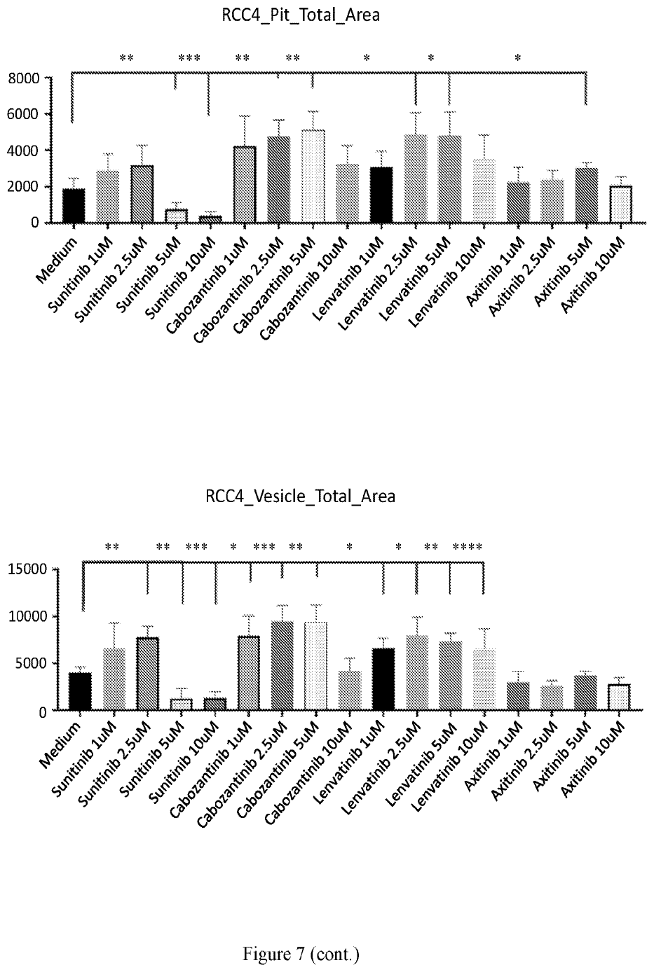

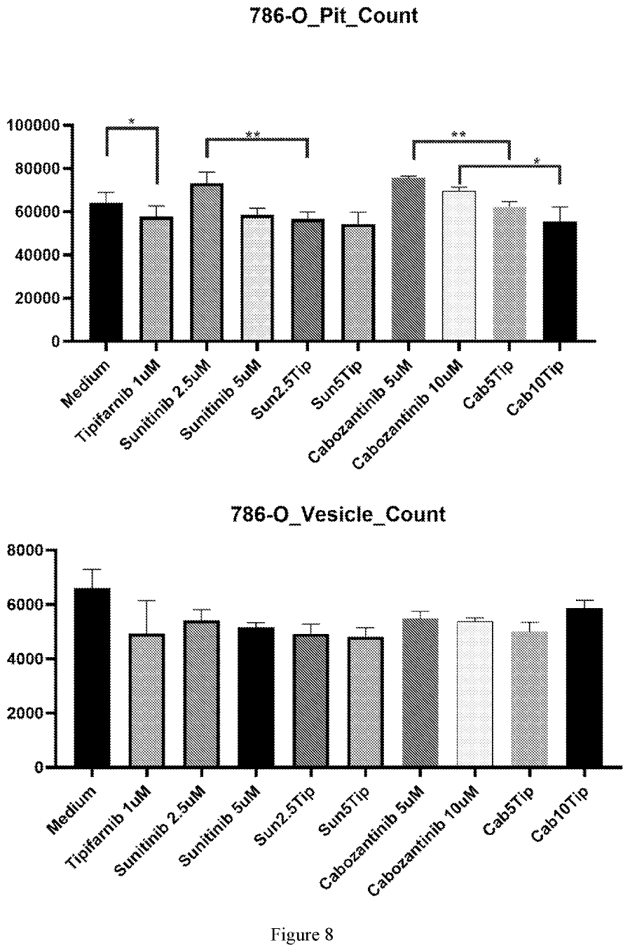

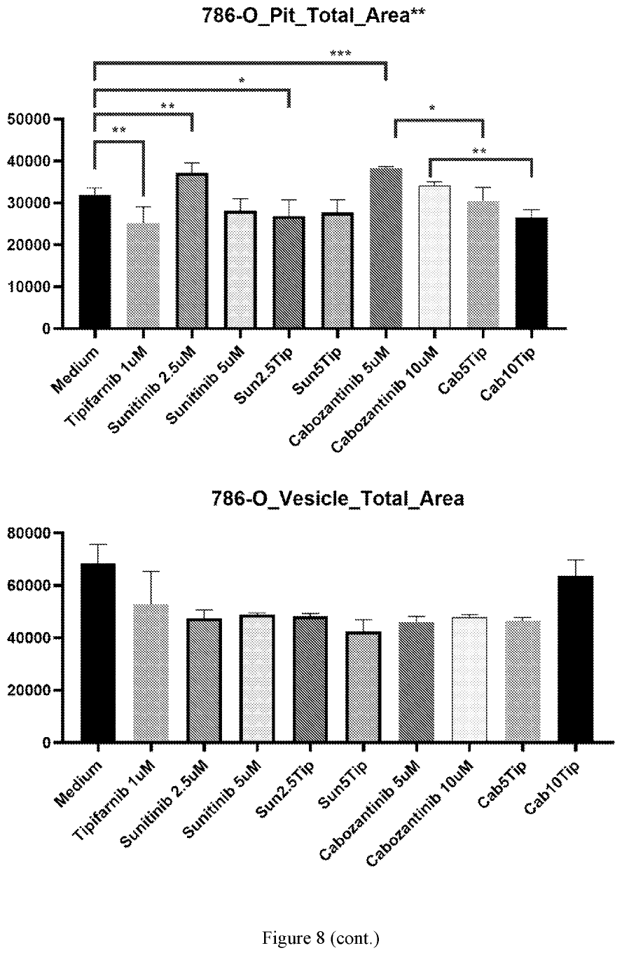

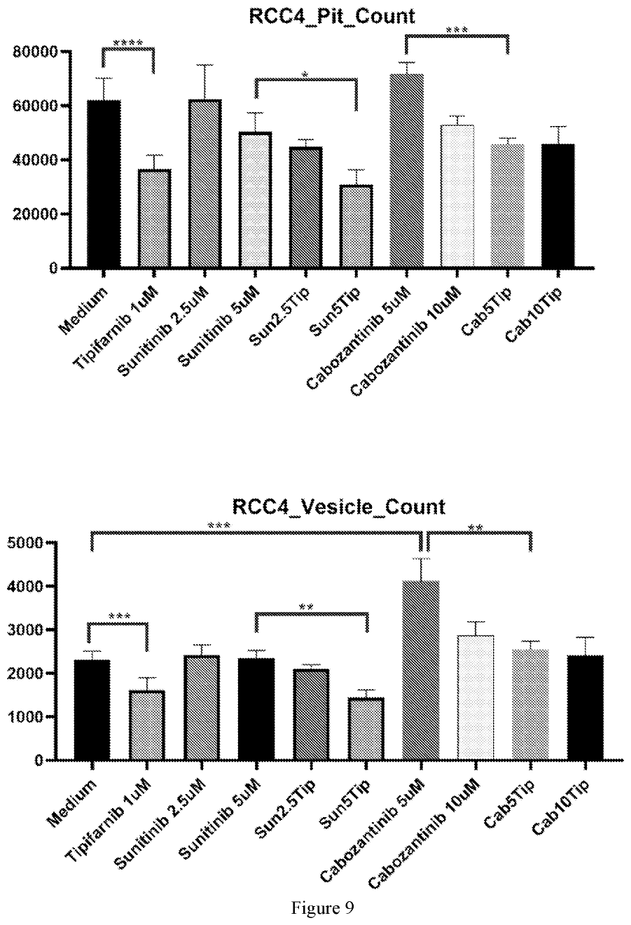

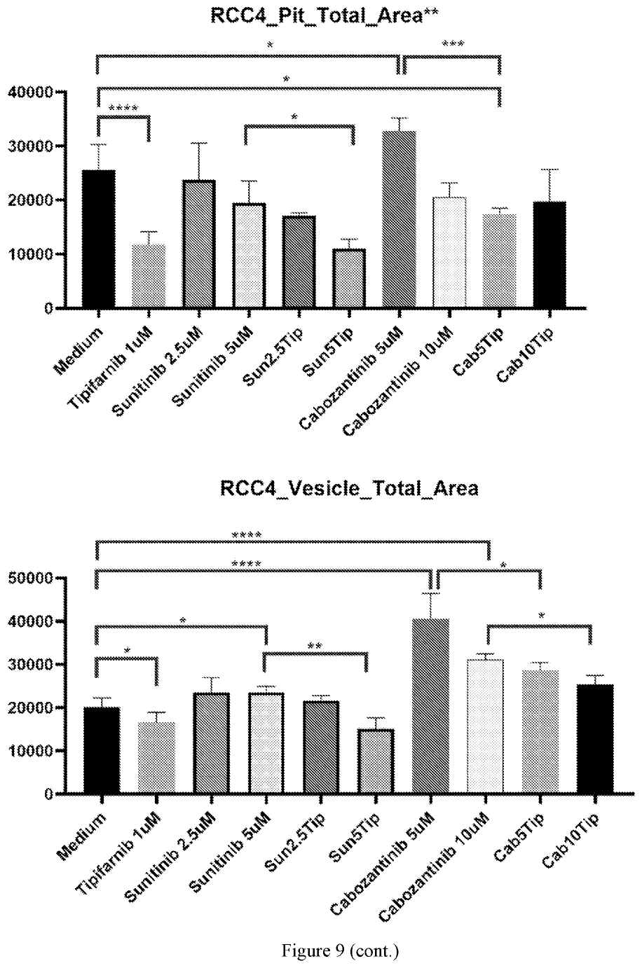

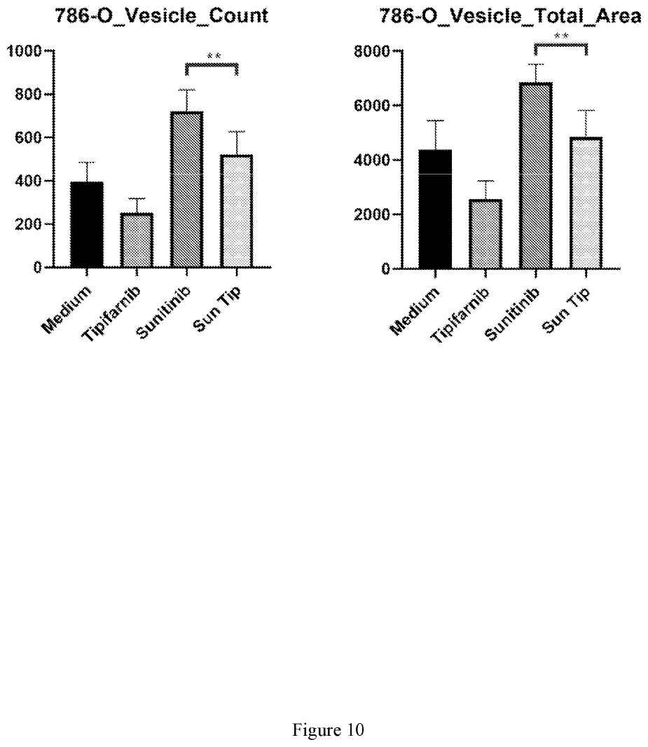

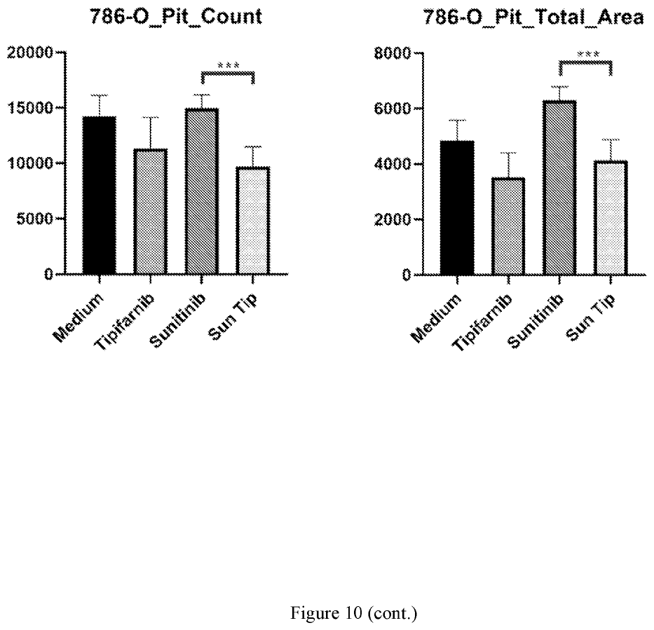

Cells were treated with escalating concentrations of tipifarnib and/or TKIs for the indicated periods of time. Image acquisition was performed on an ImageXpress micro automated imaging system (Molecular Devices, Sunnyvale, CA) driven by MetaXpress software. Nine image fields per well were acquired, using two channels to capture matching signal from Hoechst-stained nuclei (DAPI channel, ex 377/50, em 447/60) and TRITC (for LysoTracker Red, ex 525/40, em 585/40). Epifluorescence images were acquired with a 20× objective (ELWD Plan Fluor, NA 0.45, WD 7.4), using laser auto-focus with a z-offset. Images were analyzed using ‘Multiwavelength Scoring’ MetaXpress module for measurement of parameters within each fluorescent channel. Data generated from these analyses were displayed within Acuity Xpress (Molecular Devices). Representative results are shown in FIG. 5 (786-O cells), FIG. 6 (SK-45 cells), FIG. 7 (RCC4 cells), FIG. 8 (786-O cells), FIG. 9 (RCC4 cells), and FIG. 10 (786-O cells).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A pharmaceutical composition comprising:
a tyrosine kinase inhibitor chosen from cabozantinib and lenvatinib, or a pharmaceutically acceptable salt thereof:
a second agent chosen from the farnesyltransferase inhibitor tipifarnib or lonafarnib, or the Rheb inhibitor NR1, or a pharmaceutically acceptable salt thereof; and
a pharmaceutically acceptable carrier.

2. The pharmaceutical composition of claim 1, wherein the ratio of the tyrosine kinase inhibitor to the second agent is from about 0.01:1 to about 100:1 (w/w), from about 0.1:1 to about 10:1 (w/w), or from about 1:1 to about 5:1 (w/w).

3. The pharmaceutical composition of claim 1, wherein the tyrosine kinase inhibitor is present in an amount from about 1 mg to about 100 mg, from about 5 mg to about 75 mg, from about 10 mg to about 60 mg, or from about 12.5 mg to about 50 mg, and the second agent is present in an amount from about 1 mg to about 500 mg, from about 50 mg to about 400 mg, from about 75 mg to about 300 mg, or from about 100 mg to about 200 mg.

4. The pharmaceutical composition of claim 1, wherein the pharmaceutical composition is an oral dosage form, an intravenous dosage form, a topical dosage form, an intraperitoneal dosage form, or an intrathecal dosage form.

5. The pharmaceutical composition of claim 1, wherein the oral dosage form is a pill, tablet, capsule, cachet, gel-cap, pellet, powder, granule, or liquid; wherein the oral dosage form comprises about 12.5 mg, about 25 mg, about 37.5 mg, or about 50 mg of the tyrosine kinase inhibitor, and wherein the oral dosage form comprises about 25 mg, about 50 mg, about 75 mg, about 100 mg, about 150 mg, or about 200 mg of the second agent.

6. (canceled)

7. (canceled)

8. The pharmaceutical composition of claim 5, wherein the oral dosage form is a capsule.

9. The pharmaceutical composition of claim 5, wherein the oral dosage form is protected from light and present within a blister pack, bottle, or intravenous bag.

10. The pharmaceutical composition of claim 1, wherein the tyrosine kinase inhibitor is cabozantinib.

11. The pharmaceutical composition of claim 1, wherein the tyrosine kinase inhibitor is lenvatinib.

12. The pharmaceutical composition of claim 1, wherein the second agent is tipifamib, or a pharmaceutically acceptable salt thereof.

13. The pharmaceutical composition of claim 1, wherein the second agent is lonafamib, or a pharmaceutically acceptable salt thereof.

14. A method of treating cancer in a subject in need thereof comprising:
administering to the subject a tyrosine kinase inhibitor chosen from cabozantinib and lenvatinib, or a pharmaceutically acceptable salt thereof; and
administering to the subject a second agent chosen from the famesyltransferase inhibitor tipifamib or lonafamib, and the Rheb inhibitor NR1, or a pharmaceutically acceptable salt thereof.

15. The method of claim 14, wherein the ratio of the administered tyrosine kinase inhibitor to the second agent is from about 0.01:1 to about 100:1 (w/w), from about 0.1:1 to about 10:1 (w/w), or from about 1:1 to about 5:1 (w/w).

16. The method of claim 14, wherein the tyrosine kinase inhibitor is administered in an amount from about 1 mg to about 100 mg, from about 5 mg to about 75 mg, from about 10 mg to about 60 mg, or from about 12.5 mg to about 50 mg, and the second agent is administered in an amount from about 1 mg to about 500 mg, from about 50 mg to about 400 mg, from about 75 mg to about 300 mg, or from about 100 mg to about 200 mg.

17. The method of claim 14, wherein the tyrosine kinase inhibitor is administered prior to the administration of the second agent or after administration of the second agent; or wherein the tyrosine kinase inhibitor is administered concurrently with administration of the second agent.

18. (canceled)

19. The method of claim 17, wherein the tyrosine kinase inhibitor is present in a pharmaceutical composition which is an oral dosage form, an intravenous dosage form, a topical dosage form, an intraperitoneal dosage form, or an intrathecal dosage form.

20.-33. (canceled)

34. The method of claim 14, wherein the subject is also administered radiation therapy, immunotherapy, and/or neoadjuvant therapy.

35. The method of claim 14, wherein the cancer is breast cancer, pancreatic cancer, prostate cancer, lung cancer, liver cancer, esophageal cancer, stomach cancer, billary tract cancer, head and neck cancer, bladder cancer, kidney cancer, mesothelioma, thyroid cancer, uterine cancer, ovarian cancer, brain cancer, lymphoma, myeloma, leukemia, or colon cancer.

36. The method of claim 14, wherein the cancer is renal cell carcinoma, clear cell renal carcinoma (ccRCC), papillary renal cell carcinoma, chromophobe renal cell carcinoma, or unclassified renal cell carcinoma.

37. A combination of a tyrosine kinase inhibitor chosen from cabozantinib and lenvatinib, or a pharmaceutically acceptable salt thereof, and a second agent chosen from the famesyltransferase inhibitor tipifamibor lonafamib, and the Rheb inhibitor NR1, or a pharmaceutically acceptable salt thereof, for use in the manufacture of a medicament for treating cancer.

38. (canceled)
